(19) United States
(12) Patent Application Publication
(10) Pub. No.: US 2004/0219188 A1
(43) Pub. Date: Nov. 4, 2004

(54) Composition and methods for nutritional management of patients with hepatic disease

(76) Inventors: Gail M. Comer, Libertyville, IL (US); Lucia Cheng, Buffalo Grove, IL (US); Vikkie Mustad, Galena, OH (US); Yung-Sheng Huang, Upper Arlington, OH (US); John McEwen, Columbus, OH (US); Stephen J. DeMichele, Dublin, OH (US); Bruce R. Bistrian, Ipswich, MA (US); Gail M. Comer, Libertyville, IL (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/428,498

(22) Filed: May 2, 2003

(51) Int. Cl. A61K 47/00; A61K 35/78; A61K 31/70

(52) U.S. Cl. 424/439; 424/764; 424/776; 514/23

(57) ABSTRACT

This invention relates to compositions and methods for nutritional management of hepatic (liver) failure. In particular the invention is directed to a nutritionally complete formulation suitable for use as a supplement or total enteral feeding. The composition is specifically tailored to meet the requirements of hepatic patients in need of nutritional support. The composition comprises an organoleptically acceptable protein system designed to meet the altered metabolic needs of patients suffering from hepatic failure. The invention also relates to administering a nutritional composition comprising effective amounts of the branched-chain amino acids, valine, leucine, isoleucine, or mixtures thereof, and with or without a reduced amount of tyrosine, phenylalanine and tryptophan.

This invention relates to compositions and methods for nutritional management of hepatic (liver) failure. In particular the invention is directed to a nutritionally complete formulation suitable for use as a supplement or total enteral feeding in patients with liver disease. The composition is specifically tailored to meet the requirements of hepatic patients in need of nutritional support. In contrast to current hepatic formulas, the composition comprises an organoleptically acceptable protein system designed to meet the altered metabolic needs of patients suffering from hepatic failure.

### BACKGROUND

Due to a variety of insults and pathogens, the liver can become diseased. Liver disease is a broad classification encompassing a number of acute and chronic diseases. These diseases include hepatitis (viral and non-viral); cirrhosis (alcoholic and non-alcoholic); and liver failure. Liver failure is perhaps the most severe disease and may be accompanied by a complex set of conditions including hepatic encephalopathy; hemorrhage; coagulapathy; ascites; jaundice; and hepatorenal syndrome.

A proper functioning liver is of utmost importance to the survival of a patient. It is responsible for the metabolism of nearly all nutrients, and is the primary site for the inactivation of numerous toxins. The liver extracts amino acids, carbohydrates, lipids, vitamins and minerals from the portal circulation. These extracted nutrients are used as substrates or cofactors in all metabolic processes carried out in the liver.

The liver is also the site of detoxification of numerous substances, in particular those nitrogenous wastes associated with protein metabolism. The liver normally will detoxify ammonia by forming the nitrogen-containing substances urea, which is then excreted via the kidneys. When the liver is in various degrees of failure its ability to detoxify ammonia can become compromised. As a result ammonia can accumulate in the blood (hyperammonemia). Hyperammonemia has been associated with the pathogenesis of hepatic encephalopathy.

Malnutrition is recognized as a major factor in clinical outcome in chronic liver disease. The causes of malnutrition in liver disease are multifactorial and may include anorexia, nausea/vomiting, malabsorption, inadequate or unpalatable diets, medication-induced losses, micronutrient deficiencies, and altered hepatic metabolism. Protein-calorie malnutrition may be present in 20% of patients with compensated cirrhosis and up to 60% of patients with advanced decompensated disease. In addition, virtually 100% of patients with alcoholic hepatitis with or without cirrhosis are malnourished. In controlled trials, nutritional intervention that successfully increased nutrient intake was associated with a decrease in the rate of complications (ascites, gastrointestinal bleeding, encephalopathy, infection, and mortality).

Liver disease is associated with a variety of metabolic problems that affect the body's ability to handle various substrates. The majority of cirrhotic patients have impaired glucose tolerance associated with hyperinsulinemia and insulin resistance, with diabetes developing in 15% to 37%. Glycogenesis is also impaired making cirrhotic patients at risk for hypoglycemia associated with prolonged fasting. Cirrhosis patients also have increased lipid oxidation and impaired elongation and saturation of essential fatty acids. With end-stage disease, there is impaired urea synthesis with hyperammonemia and hepatic encephalopathy. Cirrhosis patients also develop excessive sodium and water retention due to secondary hyperaldosteronism.

Vitamin and mineral deficiencies are also common in chronic liver disease. Alcoholics are particularly prone to water-soluble-vitamin deficiencies, in particular B-vitamin deficiencies. Cholestatic liver diseases and alcoholic liver disease are associated with fat malabsorption and calcium, fat-soluble vitamin, and bile-salt deficiencies. The likelihood of fat soluble vitamin deficiency in cholestatic liver disease is greater with more advanced disease. Most patients with advanced liver disease require zinc supplementation, which is associated with improved taste sensation and urea synthesis. Both zinc and selenium deficiencies have been associated with impaired neurologic function and worsening of hepatic encephalopathy.

Nutritional status of hepatic patients can be improved through strategies that address specific underlying problems such as anorexia, taste and olfactory impairment, and metabolic derangements that result in inadequate macro- or micronutrient intake.

Nitrogen balance and substrate utilization in stable cirrhotic patients can be improved by modifying the patient's eating pattern from 3 meals per day to 4 to 7 small meals per day, including one late evening meal. Liquid supplements can play a key role in delivering key substrates to the patient on an oral diet thereby improving survival and hepatic function in cirrhotic patients.

A number of commercial nutritional products have been positioned on the market for hepatic patients. Amin-Hepa™ (distributed by Societe Dietetique Francaise de Fromulation et de Fabrication, Doullens, France) is a powder nutritional supplement recommended for patients with chronic liver disease. The formula provides 42% of the protein system as branched chain amino acids and a BCAA:AAA molar ratio of 15:1. The protein sources are whey protein hydrolysate, lactalbumin hydrolysate, and the free amion acids leucine, isoleucine and valine. The total protein contributes 15% of the total calories of the product while the carbohydrate and fat contribute 56% and 29% of the total calories, respectfully. When prepared at full strength, the osmolality is approximately 500 mOsm/kg.

Hepatic-Aid II® (distributed by B Braun, Bethlehem, Pa.) is a powder nutritional supplement which utilizes free amino acid protein system to provide branched chain amino acids and arginine and decreased amounts of aromatic amino acids and methionine. The formula provides 46% of the protein system as branched chain amino acids and 1.8% of the protein system as aromatic amino acids. The amino acids contribute 15% of the total calories of the product while the carbohydrate and fat contribute 57.3% and 27.7% of the total calories, respectfully. When prepared at full strength, the osmolality is approximately 560 mOsm/kg.

NutriHep™ (distributed by Clintec, Deerfield, Ill.) is ready to drink enteral nutrition designed for the hepatic patient. The whey protein/free amino acid protein system provides 50% of the protein system as branched chain amino acids, 2% of the protein system as aromatic amino acids and 11.5% of the protein system as ammonia-forming amino acids. Fat contributes 12% of total calories and 66% of the fat is MCT oil. The ratio of n6:n3 fatty acids is 4:1. The formula provides 100% of the US RDA for vitamins and minerals in 1500 Calories.

L-Emental™ Hepatic (distributed by Hormel HealthLabs, Austin, Minn.) is a powder supplement for patients with chronic liver disease. The protein system is composed of free amino acids, which provide 15% of the total calories and are enriched in branched chain amino acids (46% of the protein system). The fat provides 27.7% of the total calories and carbohydrate provides 57.3% of the total calories. When prepared as directed, the osmolality is 560 mOsm/kg of water.

Aminoleban® EN (distributed by Luen Cheong Hong LTC, Hong Kong) is formulated as an enteral nutritional containing protein, carbohydrate, fat, vitamins, minerals and trace elements to supplement insufficient nutrient intake due to reduced appetite in liver failure patents. The powder has an amino acid composition consisting of high concentrations of branched chain amino acids (85.6% of the protein system) and low concentrations of aromatic amino acids (1.1% of the protein system). The powder (50 gm) is mixed with water (150 ml) and consumed with meals three times a day.

There are also a number of U.S. patents which describe compositions for administration to patients with hepatic disease.

U.S. Pat. No. 4,898,879 to Madsen, et al. describes an amino acid composition for administration to a patient having liver disease which reduces the ammonia produced endogenously. Threonine, serine, tryptophan, glutamine, histidine and glycine are termed ammonotelic amino acids that are catabolized by the body with the release of ammonia. The formula balances the proportion of ammonotelic amino acids to other essential and nonessential amino acids. The amino acid composition is cysteine free mixture of nonessential and essential amino acids, having from 8 to 16 total mole % of the composition consisting of a combination of L-serine, L-histidine, L-thereonine, L-tryptophan, L-glutamine and L-glycine. The threonine contributing from about 2.3 to 3.9% of the total mole %. The aromatic amino acids phenylalanine tyrosine and tryptophan are present at less than 8 mole %. The branched chain amino acids leucine, isoleucine and valine are present in a total of from 40-50% of the composition by weight.

U.S. Pat. No. 4,499,076 to Ohashi, et al. describes a powder elemental diet of free amino acids, carbohydrates, fats, vitamins and minerals for liver diseases. The essential amino acids are supplemented with L-alanine, L-arginine, L-glycine, L-histidine, L-proline and L-serine. The molar ratio of (isoleucine+leucine+valine+arginine)/(phenylalanine+tyrosine+tryptophan)=50-60; the molar ratio of (isoleucine+leucine+valine+arginine)/(glycine+serine+threonine)=4-5; and the molar ratio of arginine/(glycine+serine+threonine)=0.8-1.0.

U.S. Pat. No. 3,950,529 to Fisher, et. al. described an amino acid formulation for patients with liver disease. The amino acid solution may be administered orally or intravenously. The molar ratio of (isoleucine+leucine+valine)/tryptophan=40-300; and the molar ratio of (isoleucine+leucine+valine)/(phenylalanine+tyrosine)=15-135.

U.S. Pat. No. 5,571,783 to Montagne, et al. describes a nutritionally complete composition for treating patients with hepatic disease. The composition is a nutritionally complete, calorically-dense formulation suitable for use as a supplement or total enteral feeding. The ready-to-use formula contains a protein source with at least 25% of the total protein as free amino acids. The composition contains 6 to 16% of the calories as protein, 66 to 88% of the calories as carbohydrate, and 6 to 18% as lipid. Additionally, the composition meets or exceeds 100% of the U.S. RDA for vitamins and minerals in 1000 ml of product. The amino acid profile is rich in branched chain amino acids (40 to 60% of the total amino acid content) and low in aromatic and ammonia-generating amino acids (less than 3% of the total amino acid content).

As described above, the current nutritional formulations designed to support the hepatic patient utilize high levels of free amino acids to acchieve the desired branched-chain amino acid level. Free amino acids negatively impact product stability. Equally importantly, free amino acids negatively impact the flavor of the final product. Taste is a key issue with the hepatic patient who is already dealing with problems such as anorexia and olfactory impairment. If the patient will not consume the product, they will not receive the benefits of the nutritionals designed to meet their unique nutritional needs. There is therefore a need for an improved nutritional formulation for patients with liver disease, one having improved stability and organoleptic properties.

### SUMMARY OF THE INVENTION

This invention relates to compositions and methods for nutritional management of hepatic (liver) failure. In particular the invention is directed to a nutritionally complete formulation suitable for use as a supplement or total enteral feeding. The composition is specifically tailored to meet the requirements of hepatic patients in need of nutritional support. In contrast to current hepatic formulas, the composition comprises an organoleptically acceptable protein system designed to meet the altered metabolic needs of patients suffering from hepatic failure.

The invention also relates to administering a nutritional composition comprising effective amounts of the branched-chain amino acids, valine, leucine, isoleucine, or mixtures thereof, and with or without a reduced amount of the aromatic amino acids, tyrosine, phenylalanine and tryptophan.

To this end, the present invention provides a ready-to-use formula containing an amino-nitrogen component that contains less than or equal to about 30% of the total amino-nitrogen content as branched chain amino acids. Preferably, a majority of the amino-nitrogen component is present as native, non-hydrolyzed protein. In an embodiment, greater than 75% of the amino-nitrogen component is provided as native, non-hydrolyzed protein.

In an embodiment, the invention provides a method of providing nutrition to a hepatic patient by feeding a nutrient dense formula having an amino-nitogen component from about 12 to about 20% of the total calories, a carbohydrate component from about 45 to about 65% of the total calories and a fat component from about 25 to about 35% of the total calories, wherein less than about 30% of the amino-nitrogen component is branched chain amino acids.

In another embodiment, the invention provides a method of attenuating the progression of liver disease in a hepatic patient comprising enterally administering to the patient a nutritional having a fat component containing ω-6 fatty acids and at least 5.3 g/L of ω-3 fatty acids, the weight ratio of ω-6 fatty acids to ω-3 fatty acids being from about 1.5:1 to about 5:1; and an amino-nitrogen component wherein less than 30% by weight is branched-chain amino acids, and wherein less than 15% by weight is aromatic amino acids.

In a further embodiment, the invention provides a method of correcting the nutritional deficiencies in a hepatic patient comprising enterally administering to the patient a nutritional having an fat component containing ω-6 fatty acids and at least 5.3 g/L of ω-3 fatty acids, the weight ratio of ω-6 fatty acids to ω-3 fatty acids being from about 1.5:1 to about 5:1, a amino-nitrogen component wherein less than 30% by weight is branched-chain amino acids, and aromatic amino acids are less than 15% by weight, and non-supplemented levels of iron, manganese and copper.

In a yet another embodiment, the invention provides a method for improving liver function in a hepatic patient comprising enterally administering to the patient a nutrient dense formula with FOS having a caloric distribution comprising an amino-nitogen component from about 12 to about 20% of the total calories, wherein the amino-nitrogen component contains less than about 30% as branched chain amino acids.

### DETAILED DESCRIPTION

As used herein:

The term “fatty acids” refer to a family of carboxylic acids having a hydrocarbon chain, generally from about 12 to 22 carbons long. When unsaturated (having a double bond in at least one point in the hydrocarbon chain), such fatty acids are designated by the position of the first double bond. ω-3 fatty acids have a first double bond at the third carbon from the methyl end of the chain; and include, but are not limited to, α-linolenic acid, stearidonic acid, eicosapentaenoic acid (“EPA”), docosapentaenoic acid and docosahexaenoic acid (“DHA”) and the like. ω-6 fatty acids have a first double bond at the sixth carbon from the methyl end of the chain; and include, but are not limited to, linoleic acid, γ-linolenic acid (“GLA”), arachidonic acid (“AA”), and the like. The ratio of ω-6 fatty acids to ω-3 fatty acids is simply the ratio of the total amounts (usually expressed as weight) of each type.

The term “branched-chain amino acids” (“BCAA”) refer to amino acids that have a fork or branch in the side chain. These include primarily those having a carbon-carbon branch, i.e. valine, leucine and isoleucine; but may also include other types of branches. BCAA levels are decreased in the blood of cirrhosis patients. Additionally, BCAA have several properties of potential benefit to patients with chronic liver disease including: inhibition of protein breakdown; increasing the synthesis of hepatic and muscle protein; and serving as an energy source for skeletal muscle. BCAA can also be used for gluconeogensis, particularly in skeletal muscle.

The term “aromatic amino acids” (“AAA”) refer to amino acids that have an aromatic ring in the side chain. These primarily include tyrosine, phenylalanine and tryptophan. MA levies are increased in the blood of cirrhotic patients and have been associated with increased hepatic encephalopathy.

The term “amino-nitrogen component” is utilized herein interchangably with free amino acids, intact or native protein, pepetides and hydrolyzed protien. Typically amino-nitrogen refers to any or a combination of the following: free amino acids, intact or native protein, pepetides and hydrolyzed protien. The amino-nitrogen component will typically be composed of a mixture of native protein and free amino acids.

“Nutritional matrix” as used herein refers to a delivery vehicle that contains fats, amino-nitrogen and carbohydrates and provides some or all of the nutritional support for a patient in the recommended daily amounts. Frequently a nutritional matrix will contain vitamins, minerals, trace minerals and the like to provide balanced nutrition.

Nutritional support in the hepatic patient can be categorized as (i) supportive, in which nutrition support is instituted to prevent nutrition deterioration in the adequately nourished patient or to rehabilitate the depleted patient before definitive therapy; (ii) adjunctive, in which nutrition support plays an integral role in the therapeutic plan; and (iii) definitive, in which aggressive nutrition support is required for the patient's existence. The routes for providing nutrition support include an oral diet, enteral tube feeding and total parenteral nutrition. The preferred route of administration for nutritional methods and compositions of the invention is by the oral route. An alternate to oral feeding is enteral tube feeding by means of nasogastric, nasoduodenal, esophagostomy, gastrostomy, or jejunostomy tubes.

A typical nutritional composition useful in this invention will have a caloric distribution as follows: from about 12 to about 20% from the amino-nitrogen component, from about 45 to aboout 65% from the carbohydrate component and from about 25 to about 35% from the fat component. In another embodiment, the nutritional composition comprises a caloric distribution of about 12 to about 18% from the amino-nitrogen component, about 45 to about 60% from the carbohydrate component and about 25 to 33% from the fat component. In yet another embodiment, the nutritional composition comprises a caloric distribution of about 14 to about 18% from the amino-nitrogen component, about 50 to about 58% from the carbohydrate component and about 27 to about 33% from the fat component.

The liver has an essential role in the synthesis, regulation, and metabolism of lipids. It is responsible for the elongation and desaturation of essential fatty acids to form long-chain polyunsaturated fatty acids, some of which are precursors for prostaglandin formation. However, patients with chronic liver disease may develop fatty acid deficiencies due to the inability to elongate or desaturate the essential fatty acids, linoleic and linolenic. Malnutrition is a major risk factor for this impaired lipid unsaturation in cirrhosis. For this reason, fatty acid metabolites of these essential fatty acids become conditionally essential. Further, due to the impaired elongation and desaturation of linoleic acid, liver disease patients have been shown to have an altered ratio of linoelic to arachidonic acid, and a decreased proportion of phospholipid and cholesterol ester arachidonic acid. Consequently, arachidonic acid has been considered “conditionally essential” in advanced liver disease. Additonally, arachidonic acid deficiency has been associated with increased mortality risk in patients with advanced cirrhosis.

Gamma-linolenic acid (GLA) and eicosapentaenoic acid (EPA) are competitive inhibitors of cyclooxygenase, the major enzyme of arachidonic acid metabolism. By feeding both GLA and EPA, there is competitive inhibition of cyclooxygenase and down-regulation of proinflammatory arachidonic acid metabolites. Therefore, by feeding oils rich in GLA, EPA, and DHA, not only are the essential fatty acid deficiencies (GLA, EPA, and DHA) corrected but there is an anti-inflammatory effect as well.

These conditionally essential fatty acids are supplemented in nutritional of the instant invention by the incorporation of oils rich in gamma-linolenic acid, dihomo-gamma-linolenic, eicosapentaenoic acid, docosahexanoic acid and arachidonic acid. Examples of source oils rich in the above fatty acids include but are not limited to borage oil, evening primrose oil, black currant oil, fungal and algael oil, which provides gamma-linolenic acid (GLA) and dihomo-gamma-linolenic (DGLA); marine oils such as mackerel, sardine, menhadin, anchovy, herring, which provide eicosapentaenoic acid (EPA) and docosahexanoic acid (DHA); and fungal oil and algael oil, which provides arachidonic acid (AA). U.S. Pat. No. 5,492,938 to Kyle et al. describes a method of obtaining DHA from dinoflagellates and its use in dietary supplements. Additonally, DHA is available from Martek Biosciences Corporation of Columbia, Md. Arachidonic acid is available from Genzyme Corporation of Cambridge, Mass. Algal oils such as those from dinoflagellates of the class Dinophyceae, notably Crypthecodinium cohnii are also sources of DHA (including DHASCO™), as taught in U.S. Pat. Nos. 5,397,591, 5,407,957, 5,492,938, and 5,711,983. The genus Mortierella, especially M. alpina, and Pythium insidiosum are good sources of AA, including ARASCO™ as taught by U.S. Pat. No. 5,658,767 and as taught by Yamada, et al. J. Dispersion Science and Technology, 10(4&5), pp561-579 (1989), and Shinmen, et al. Appl. Microbiol. Biotechnol. 31:11-16 (1989).

Table 1 sets forth both example levels and typical ranges for conditionally essential fatty acids useful in the nutritional of the invention. Typically, the weight ratio of ω-6 fatty acids to ω-3 fatty acids in the lipid blend according to the invention is from about 1.5:1 to 5:1. In another emboidiment, the weight ratio of ω-6 fatty acids to ω-3 fatty acids is from about 1.5:1 to 3:1. In yet another embodiment, weight ratio of ω-6 fatty acids to ω-3 fatty acids if from about 2:1 to 3:1.

TABLE 1
Conditionally Essential Fatty Acids

(gm/100 Kcal)

| Fatty Acid | Example | Typical Range |
|---|---|---|
| EPA | 0.19 | 0.1-0.4 |
| GLA | 0.19 | 0.1-0.4 |
| DHA | 0.082 | 0.03-0.3 |
| AA | 0.066 | 0.03-0.3 |

As described above, the fat component contributes from about 25 to about 35% of the total calories of the nutritional. More particularly, the fat component is in compliance with American Heart Association guidelines that limit the saturated fat (FSA) and polyunsaturated fat (PUFF) each to less than 10% of total calories. Table 2 sets forth example levels and typical ranges of a typical fat component useful in the nutritional of the invention.

TABLE 2
Typical Fat Component

(% total weight of fat component)

| OIL | Example | Typical Range |
|---|---|---|
| High oleic sunflower oil | 24% | 15-30% |
| MCT | 20% | 15-30% |
| Borage | 26% | 20-35% |
| Fish | 21.5% | 15-30% |
| Fungal | 4.5% | 3-6% |
| Soy lecithin | 4.0% | 3-5% |

Numerous commercial sources for the fats listed above are readily available and known to one practicing the art. For example, fractionated coconut oil (MCT) is available from Henkel Corporation of LaGrange, Ill. High oleic sunflower oil is available from SVO Specialty Products of Eastlake, Ohio. Fish oil is available from Mochida International of Tokyo, Japan. Borage oil is available from PGE Canada of Bioriginal Food Science Corporation, Saskatoon, Sascachewan.

Table 3 presents a typical fatty acid profile of an exemplary oil component useful in the present invention. The weight ratio of the total ω-6 fatty acids to the total ω-3 fatty acids in this embodiment is 2.35 which is within the claimed range for this invention.

TABLE 3
Typical Fatty Acid Profile

| | (% of total fatty acids by weight) |
|---|---|
| Fatty Acid | gm/100 gm fat |
| Caproic (6:0) | 0.02 |
| Caprylic (8:0) | 12.80 |
| Capric (10:0) | 7.10 |
| Lauric (12:0) | 0.08 |
| Myristic (14:0) | 1.31 |
| Palmitic (16:0) | 6.61 |
| Palmitolaic (16:1w7) | 2.03 |
| Stearic (18:0) | 1.72 |
| Oleic (18:1w9) | 28.17 |
| Linoleic (18:2w6) | 16.77 |
| Gamma-Linolenic (18:3w6) | 6.09 |
| Alpha-linolenic (18:3w3) | 0.51 |
| Stearidonic (18:4w3) | 0.66 |
| Arachidic (20:0) | 0.21 |
| Eicosadienoic (20:2w6) | 0.02 |
| Arachidonic (204w6) | 2.09 |
| Eicosapentaenoic (20:5w3) | 6.02 |
| Docosapentaenoic (22:5w3) | 0.67 |
| Docosahexaenoic (22:6w3) | 2.58 |
| Others | 4.33 |

Table 4 sets forth selected characteristics of a typical fat component useful in this invention. However, it will be realized that the characteristics may vary among other formulas useful for this invention, depending on the specific fat sources added and the ratios in which they are used.

TABLE 4
Typical Fat Blend Characteristics

| Characteristic | Example |
|---|---|
| ω-3 fatty acids, gm/100 gm fat | 10.64 |
| ω-6 fatty acids, gm/100 gm fat | 24.97 |
| saturated fatty acids, gm/100 gm fat | 29.84 |
| monounsaturated fatty acids, gm/100 gm fat | 32.37 |
| polyunsaturated fatty acids, gm/100 gm fat | 37.78 |
| ω-6/ω-3 ratio | 2.35:1 |
| polyunsaturated fatty acids, % total calories | 11.33 |
| monounsaturated fatty acids, % total calories | 9.71 |
| saturated fatty acids, % total calories | 8.95 |

Diphosphatidyl choline may optionally be added to the nutritional formula of the invention in levels from about 0 to about 1.5 gm/L of the nutritional product. In another embodiment, diphosphatidyl choline may be added to the nutritional formula in levels from about 0.5 to about 1.5 gm/L of the nutritional product. In yet another embodiment, diphosphatidyl choline may be added to the nutritional formula in levels from about 0.5 to about 1.0 gm/L of the nutritional product. At effective levels, diphosphatidyl choline has been shown to reduce inflammation in alcoholic liver disease via its antioxidant properties. Further, choline deficiency has been associated with the development of fatty liver. Sources of diphosphatidyl choline include, but are not limited to, soy lecithin and egg yolk lecithin. Commercial suppliers of lecithin include Archer Daniels Midland Company of Decatur, Ill.; Central Soya Company of Fort Wayne, Ind.; Pfanstiehl Laboratories of Waukegan, Ill. and Lucas Meyer of Decatur Ill.

A second component of the nutritional product is the amino-nitrogen component. Protein is needed to increase lean body mass. The required protein intake varies with disease severity but most patients with cirrhosis can tolerate 0.8-1.0 g/kg, and well-compensated patients may tolerate up to 1.5 g/kg. Patients with mild encephalopathy can be transiently fed as low as 0.5 g/kg of protein. As described above, the amino-nitrogen component contributes from about 12 to about 20% of the total calories of the nutritional of the invention.

BCAA (leucine, valine and isoleucine) stores tend to decrease with liver failure because they are a source of energy for skeletal muscle, heart, and brain when gluconeogenesis and ketogenesis are depressed. BCAAs have several properties of potential benefit to patients with chronic liver disease. BCAAs inhibit protein breakdown and increase the synthesis of hepatic and muscle proteins and serve as an energy source via glycogenesis in skeletal muscle. Leucine is the most important determinant of nitrogen sparing rather than the total amount of BCAAs.

The aromatic amino acids (AAAs) tyrosine, phenylalanine, and tryptophan blood levels are increased in cirrhotic patients and have been associated with increased hepatic encephalopathy due to cerebral uptake and increased cerebral serotonin and catecholamines. The resulting alteration in the plasma molar ratio of BCAA:AAA has been suggested as an etiologic factor of hepatic encephalopathy. Correction of this abnormal ratio is associated with improvement in hepatic encephalopathy.

The amino-nitrogen sources that are useful for the nutritional products of the invention include any amino-nitrogen sources that are suitable for human consumption. Such amino-nitrogen sources are well known by those skilled in the art and can be readily selected when preparing such products. Examples of suitable amino-nitrogen sources typically include casein, whey, milk, soy, pea, rice, and corn protein in their native and/or hydrolyzed form, free amino acids, glycomacropeptide and mixtures thereof.

Commercial protein sources are readily available and known to one practicing the art. For example, caseinates, whey, hydrolyzed caseinates, hydrolyzed whey and milk proteins are available from New Zealand Milk Products of Santa Rosa, Calif. Soy and hydrolyzed soy proteins are available from Protein Technologies International of Saint Louis, Mo. Pea protein is available from Feinkost Ingredients Company of Lodi, Ohio. Rice protein is available from California Natural Products of Lathrop, Calif. Corn protein is available from EnerGenetics Inc. of Keokuk, Iowa.

The amino-nitrogen source is typically an intact protein (native, non-hydrolyzed) of high biologic value, inherently high in branched chain amino acids (BCAA), low in aromatic amino acids (AAA) and palatable. One of the most common biological methods for evaluating the nutritional value of proteins is the protein efficiency ratio (PER). The PER shows how well test animals utilize protein by measuring their weight gain on a controlled diet. The more weight gain per unit of protein intake, the higher the PER of the tested protein. Milk proteins rank higher on the PER scale when compared to vegetable proteins. For example, the PER for: whey protein concentrate is 3.0; lactalbumin is 2.9; milk protein isolate is 2.8; casein is 2.5; rice is 2.2; soy protein isolate is 1.8; and wheat gluten is 1.1. Table 5 lists the BCAA and AAA content of several different amino-nitrogen sources.

TABLE 5
BCAA and AAA Content of Different Protein Sources*
(gm/100 gm protein)

| | BCAA | | | AAA | | |
|---|---|---|---|---|---|---|
| Protein | Isoleucine | Leucine | Valine | Phenylalanine | Tryptophane | Tyrosine |
| Casein | 5.63 | 9.22 | 6.9 | 4.89 | 1.20 | 6.3 |
| Whey | 6.12 | 12.9 | 5.82 | 3.59 | 1.72 | 3.2** |
| Milk, dry skim | 6.12 | 10.35 | 6.93 | 4.54 | 1.12 | 5.1** |
| Soybean flour | 4.98 | 7.49 | 4.9 | 4.76 | 1.12 | 4.3 |
| Whole wheat | 3.60 | 5.89 | 4.10 | 4.06 | 0.83 | 3.0 |

*Data from Introductory Food Chemistry, Ira Garard, Ph.D, The AVI Publishing Company, Inc. Westport, Connecticut, 1978 and World Protein Resources, Allen Jones, John Wiley & Sons, New York, 1974
**Tyrosine data from Swaisgood HE, in Handbood of Milk composition, 1995, Academic Press

Examples of high quality amino-nitrogen sources containing high BCAA typically include casein, whey and their partially hydrolyzed forms, and glycomacropeptide. While free BCAA are the simplest source to utilize to obtain the preferred BCAA concentration, they do not contribute to the physical stability of a nutritional formula and they have a bitter taste that contributes to a less palatable nutritional product. To this end, the present invention provides a nutritional formula containing an amino-nitrogen component that contains a majority of native, non-hydrolyzed form of amino-nitrogen. In an embodiment, at least 75% of the amino-nitrogen component is provided as native, non-hydrolyzed protein, with the balance of amino-nitrogen component comprising free amino acids and/or hydrolyzed protein.

Typically, the amino-nitrogen component comprises branched chain amino acids (BCAA) at less than about 30 wt/wt % of the total amino-nitrogen and aromatic amino acids (AAA) from about 5 to about 15 wt/wt % of the total amino-nitrogen. In another embodiment, the BCAA contribute from about 20 to about 27 wt/wt % and AAA contribute from about 7 to about 13 wt/wt % of the total amino-nitrogen. The typical amino-nitrogen component has a BCAA:AAA ratio of from about 1.5:1 to 5:1. In another embodiment, the amino-nitrogen component has a BCAA:AAA ratio of from about 1.5:1 to 4:1

An example of an aceptable amino acid profile utilizing a typical amino-nitrogen component comprising 83 wt/wt % casein, 10 wt/wt % whey and 7 wt/wt % leucine is presented in Table 6.

TABLE 6
Typical Amino Acid Profile

| | g/100 g amino-nitrogen |
|---|---|
| Essential Amino Acids | |
| Histidine | 2.25 |
| Isoleucine | 4.28 |
| Leucine | 15.51 |
| Lysine | 6.85 |
| Methionine | 2.28 |
| Phenylalanine | 4.38 |
| Threonine | 4.19 |
| Tryptophan | 1.07 |
| Valine | 5.39 |
| Arginine | 2.98 |
| Nonessential Amino Acids | |
| Alanine | 2.91 |
| Aspartic acid | 6.89 |
| Cystine | 0.58 |
| Glutamic acid | 19.56 |
| Glycine | 1.76 |
| Proline | 9.38 |
| Serine | 5.21 |
| Tyrosine | 4.52 |
| Total AAA | 9.97 |
| Total BCAA | 25.18 |
| BCAA:AAA | 2.52:1 |

A third component of the nutritional product is a source of carbohydrate. Because patients with chronic liver disease have an increased incidence of glucose intolerance, the carbohydrate component is limited to about 45 to about 65% of the total calories of the nutritional.

Suitable carbohydrates include, but are not limited to, hydrolyzed, intact, naturally and/or chemically modified starches sourced from corn, tapioca, rice or potato in waxy or non waxy forms; and sugars such as glucose, fructose, lactose, sucrose, maltose, high fructose corn syrup, corn syrup solids, fructooligosaccharides, and mixtures thereof.

Fructo-oligosaccharides (FOS) and other oligosaccharides provide multiple beneficial effects that are particularly useful in patients with chronic liver disease. First, FOS improve bowel function by decreasing constipation and diarrhea. Constipation can worsen hepatic encephalopathy so it should be avoided. Secondly, FOS increase the growth of beneficial bacteria (Bifidobacter) in the colon that ferment FOS to produce short-chain fatty acids. The short-chain fatty acids are reabsorbed and metabolized. This in turn lowers the pH and makes the environment less favorable to pathogenic bacteria. This bacterial fermentation of FOS also leads to the elimination of ammonia via the gut in a mechanism similar to that of lactulose.

FOS may be added to the nutritional formula at a level from about 0 to about 15 gm/liter of the nutritional. In another embodiment, the FOS comprises from about 5 to about 15 gm/liter of the nutritional. FOS is available from Golden Technologies Company, Inc, Golden, Colo.

The nutritional formulas preferably contain vitamins and minerals in an amount designed to supply or supplement the daily nutritional requirements of the person receiving the formula. Those skilled in the art recognize that nutritional formulas often include overages of certain vitamins and minerals to ensure that they meet targeted level over the shelf life of the product. These same individuals also recognize that certain micronutrients may have potential benefits for people depending upon any underlying illness or disease that the patient is afflicted with. For example, hepatic patients benefit from such nutrients as vitamin A, vitamin E, vitamin C, vitamin D, vitamin K, water soluble vitamins; and the minerals zinc, calcium, magnesium, selenium, chromium and molybdenum. Likewise there are minerals, such as iron, copper and manganese that should be limited in a hepatic patient's diet due to abnormal accumulation in the body. Nutritionals of this invention typically include, but are not limited to, the following vitamins and minerals: calcium, phosphorus, sodium, chloride, magnesium, zinc, selenium, iodine, chromium, molybdenum, carnitine, taurine, and Vitamins A, C, D, E, K and the B complex, and mixtures thereof.

In an embodiment of this invention, the nutritional product provides at least 100% of the U.S. RDA for the typical vitamins and minerals listed above, with the exception of iron, copper and manganese, in 1000 mL, which would provide 1500 kcal per day.

The vitamin/mineral system of the nutritional of the instant invention typically comprises antioxidants such as vitamin A, carotenoids, vitamin E, vitamin C and selenium. Antioxidants have beneficial effects of reducing the amount of free radicals, which are an important cause of liver injury in chronic hepatitis.

Vitamin A and alpha- and beta-carotene levels are lower in patients with liver disease than in control populations. The level of vitamin A should not exceed the RDA since it accumulates in the liver and is therefore a source of concern in liver patients. However, beta-carotene may be added in its place because beta-carotene does not metabolize to vitamin A unless it is necessary, thereby alleviating the liver toxicity issues. Vitamin E plasma and liver levels are decreased in patients with chronic liver disease. These levels are even lower in cirrhosis patients with hepatocellular carcinoma. Vitamin E also plays a role in reducing lipid peroxidative damage in the liver from carbon tetrachloride or galactosamine. Additionally, vitamin E acts as an antioxidant in the nutritional formula reducing the oxidative damage which can result from the polyunsaturated fatty acids in the liquid products.

A representative antioxidant profile useful in the nutritional of the invention is presented in Table 7 with typical range values and an examplary embodiment.

TABLE 7
Typical Antioxidant Profile

| Antioxidant | Example | Typical Range |
|---|---|---|
| Beta-carotene | 800 μg/L | 390-1200 μg/L |
| Vitamin E | 400 IU/L | 195-600 IU/L |
| Vitamin C | 400 mg/L | 195-600 mg/L |
| Selenium | 76 μg/L | 20-90 μg/L |

An example of an overall nutrient profile is set forth in Table 8.

TABLE 8
Nutrient Profile Example

| Nutrient | Qty/Liter |
|---|---|
| Protein, g | 60 |
| Fat, g | 50 |
| Carbohydrate, g | 193 |
| Soy Lecithin, g | 2 |
| FOS, g | 10 |
| Beta-carotene, μg | 800 |
| Vitamin A, IU | 1701⁺ |
| Vitamin D, IU | 600 |
| Vitamin E, IU | 400 |
| Vitamin K, μg | 225 |
| Vitamin C, mg | 400 |
| Folic Acid μg | 675 |
| Thiamine, mg | 3.6 |
| Riboflavin, mg | 3.6 |
| Vitamin B₆, mg | 5 |
| Vitamin B₁₂, μg | 7.5 |
| Niacin, mg | 30 |
| Choline, mg | 635 |
| Biotin, μg | 64 |
| Pantothenic Acid, mg | 15 |
| Sodium, mg | 500 |
| Potassium, mg | 1400 |
| Chloride, mg | 500 |
| Calcium, mg | 1200 |
| Phosphorous, mg | 900 |
| Magnesium, mg | 300 |
| Iodine, μg | 158 |
| Copper*, mg | Not detectable |
| Manganese*, mg | 0.2 |
| Zinc, mg | 50 |
| Iron*, mg | 1.8 |
| Selenium, μg | 76 |
| Chromium, μg | 120 |
| Molybdenum, μg | 100 |
| Carnitine, mg | 120 |
| Taurine, mg | 300 |
| Kcal/mL | 1.5 |

⁺Value includes 800 ug of beta-carotene.
*These minerals are not fortified, the level present is dependent on inherent levels that are supplied by the other ingredients.

The nutritional formulas may also contain a flavor to enhance its palatability. Useful flavorings include, but are not limited to chicken, orange, peach, toasted almond amaretto, wafer, melon, caramel cinnamon, banana, vanilla cookie, and coffee. Artificial sweeteners may be added to complement the flavor and mask bitter taste. Useful artificial sweeteners include saccharin, sucralose, and acesulfane-K (ace-K).

The energy density of the nutritional composition when in liquid form, can typically range from about 0.5 to 2 Kcal per ml.

Nutritional formulas can be manufactured using techniques well known to those skilled in the art. Various processing techniques exist. Typically these techniques include formation of a slurry from one or more solutions which may contain water and one or more of the following: carbohydrates, proteins, lipids, stabilizers, vitamins and minerals. The slurry is emulsified, homogenized and cooled. Various other solutions may be added to the slurry before processing, after processing or at both times. The processed formula may be packaged in a concentrated liquid form, dried to a powder form or diluted to a ready-to-feed form. The formula may then be packaged in any form that is desirable to the consumer or health care practitioner.

In another embodiment, the invention provides a method for correcting the nutritional deficiencies of a hepatic patient by adiministering the nutritonal of the instant invention which comprises a unique amino acid and fatty acid profile, high levels of micronutrients, and no supplemental iron, mangnaese and copper to reduce toxicity symptoms.

In yet another embodiment, the invention provides a method for improving nutrient intake of a hepatic patient by administering the nutritonal of the instant invention which comprises a high caloric, nutrient dense formula with an appropriate caloric distribution for better utilization of energy and a wide range of essential as well as conditionally-essential micronutrients. Additonally, the adequate levels of protein with branched chain amino aicds helps to improve nitrogen balance, serum protein conentrations and anthropometric measures A further embodiment of the invention comprises a method for attenuating the progression of liver disease by administering the nutritional of the instant invention which comprises a unique fat component that is high in omega-3 fatty acids to minimize the imflammatroy process and phosphatidylcholine to help prevent the formation of fibrosis. Additonally, the provision of effective levles of branched chain amino aicds and limited levels of aromatic amion acids enables protein-intolerant patients to attain positive nitrogen balance without increasing the risk of hepatic encephalopathy.

Another embodiment of the invention comprises a method for imporving liver function by administering the nutritional of the instant invention which comprises adequate levies of protein to support liver regeneration, high biological value protein to reduce the formation of ammonia and FOS to help excrete blood ammonia in the stool. Additonally, adequate calories help to avoid protein breakdown and MCT aids fat digestion and absorption which reduces stool fat.

The following examples are set forth to illustrate various embodiments of the invention but the scope of the invention is defined by the appended claims.

### EXAMPLE I

The list of materials for manufacturing the nutritional product of this Example I is presented in Table 9. Of course, various changes in specific ingredients and quantities may be made without departing from the scope of the invention.

TABLE 9
Bill Of Materials

| Ingredient | Amount per 1000 kg | |
|---|---|---|
| Maltodextrin | 191.3 | kg |
| Caseinate | 48.9 | kg |
| Borage oil | 11.6 | kg |
| High oleic sunflower oil | 10.7 | kg |
| fructooligosaccharide | 10.4 | kg |
| Marine oil | 9.6 | kg |
| Fractionated coconut oil | 8.9 | kg |
| Whey protein concentrate | 7.0 | kg |
| L-leucine | 3.8 | kg |
| Potassium citrate | 2.9 | kg |
| Arachidonic oil | 2.0 | kg |
| Lecithin | 1.8 | kg |
| Magnesium phosphate | 1.7 | kg |
| Sodium citrate | 1.2 | kg |
| Tricalcium phosphate | 1.0 | kg |
| Ascorbic acid | 910.0 | gm |
| Choline chloride | 661.4 | gm |
| Alpha tocopheryl acetate | 435.6 | gm |
| Potassium hydroxide | 351.3 | gm |
| Taurine | 290.0 | gm |
| Zinc sulfate | 210.0 | gm |
| Sucralose | 156.3 | gm |
| Carnitine | 115.0 | gm |
| Gellan gum | 95.0 | gm |
| Calcium pantothenate | 21.7 | gm |
| Beta carotene | 6.1 | gm |
| Niacinamide | 20.0 | gm |
| Thiamine Hydrochloride | 6.2 | gm |
| Pyridoxine hydrochloride | 6.0 | gm |
| Vitamin A palmitate | 4.8 | gm |
| Riboflavin | 4.8 | gm |
| Sodium bicarbonate | 1.4 | gm |
| Folic acid | 900.0 | mg |
| Vitamin D | 800.0 | mg |
| Chromium chloride | 678.0 | mg |
| Phylloquinone | 280.0 | mg |
| Sodium molybdate | 230.0 | mg |
| Sodium selenate | 207.0 | mg |
| Potassium iodide | 179.0 | mg |
| Biotin | 85.0 | mg |
| Cyanocobalamin | 6.5 | mg |

The liquid nutritional product of the present invention may be manufactured by a 1-kettle process. The process for manufacturing 1000 Kgs of the liquid nutritional product, using the List of Materials from Table 9, is described in detail below.

The oil slurry is prepared by combining and heating in a separate tank the MCT, borage oil and high oleic sunflower oil to a temperature in the range of about 37 to 49° C. with agitation. The vitamin A, vitamin E, vitamin D3, phylloquinone and beta-carotene are added to the oils with agitation.

For a 1,000 kg batch about 455 kg water of 67 to 71° C. is added to the blend tank. Gellan gum is dissolved together with potassium citrate in water of 70 to 75° C. and added to the blend tank. The minerals potassium chloride, sodium citrate and magnesium phosphate are dissolved in water of 70 to 75° C. and added to the blend tank. Zinc sulfate is added to the blend tank after dissolving in water of 30 to 40° C. and added to the blend tank. Potassium iodide, chromium chloride, sodium molybdate and sodium selenate are added to the blend tank after dissolving in water of 30 to 40° C. and added to the blend tank. Under continuous mixing and recirculating using a mixing apparatus leucine and calcium caseinate are dissolved and added to the blend tank. Under continuous mixing and recirculating using a mixing apparatus maltodextrin, whey protein, fructooligosaccharide, tricalcium phosphate are dissolved and added to the blend tank. Lecithin is dissolved in water of 70 to 75° C. and added to the blend tank. The oil slurry containing the oil soluble vitamins is added to the blend tank. The resultant blended slurry is maintained at a temperature in the range 55 to 65° C. for no longer than 3 hours.

The pH of the slurry is determined after 15 minutes agitation and if necessary is adjusted with diluted potassium hydroxide if it is outside the range of 6.70-6.95.

After waiting a period of not less than 10 minutes the blended slurry is homogenized, heat treated during 5 seconds at 145° C., cooled and stored in a product storage tank under continuous agitation at a temperature of 4 to 10° C.

The marine oil and arachidonic acid oil are slowly metered into the product as the blend passes through the homogenizer at a constant rate. At this time appropriate analytical testing for quality control is conducted. Based on the test results an appropriate amount of dilution water is added to the homogenized slurry with agitation. The artificial sweetener sucralose solution is prepared by dissolving in water of 30 to 40° C. and then adding to the homogenized slurry.

The water soluble vitamin solution is prepared by using about 4 kg of water to a temperature in the range of about 20 to 30° C. with agitation, and thereafter adding the following ingredients, in the order listed: niacinamide, calcium pantothenate, thiamine HCl, pyridoxine HCl, riboflavin, biotin, sodium bicarbonate, folic acid and vitamin B12. The vitamin solution is then added to the blended slurry with agitation.

Next the taurine and L-carnitine are added to about 8 kg of water of 48 to 60° C. and dissolved by agitating. The solution is added to the homogenized slurry with agitation.

To standardize the slurry potassium hydroxide, ascorbic acid and choline chloride are dissolved in water of 20 to 30° C. and added to the slurry.

Finally a flavor solution is prepared by adding the flavor to about 3 kg of water of 20 to 30° C. with agitation. A nutritional product according to the present invention has been manufactured using the following flavors based on the finished product weight: 1.25% natural chicken; 0.3% N&A orange; 0.2% artificial peach; 0.05% artificial toasted almond amaretto; 0.35% natural wafer; 0.25% artificial melon; caramel cinnamon (made by combining 0.06% natural vanilla coconut, 0.1% natural cinnamon, and 0.15% natural caramel flavors); banana (made by combining 0.06% nature identical banana, 0.12% natural caramel); vanilla cookie (made by combining 0.1% artificial butter, 0.1% artificial pecan); coffee (combining 0.5% natural coffee, 0.04% artificial coffee). The flavor solution is then added to the blended slurry with agitation.

If necessary, diluted potassium hydroxide is added to the blended slurry such that the final product will have a pH in the range of 6.50 to 6.90 after sterilization.

In case of a tetra pack presentation, the completed product is aseptic processed and filled aseptically in to desired containers.

In case of a can presentation, the completed product is placed in suitable containers and subjected to terminal sterilization.

### EXAMPLE II

The objective of this experiment was to evaluate the organoleptic characteristics of different flavored nutritional composition of the invention and two commercially available products marketed to the hepatic patient. To measure organoleptic properties, taste standards, described in Table 10, were prepared to rank the sweet, bitter and sour intensity of the test compositions.

TABLE 10
Taste Intensity Scale References

| | Basic | | Concentration* | Representative |
|---|---|---|---|---|
| Standard | Taste | Intensity | by Weight | Products |
| 1 | Sour | 1 | 0.05% Citric Acid | Milk Chocolate, |
| | | | | Coffee |
| 2 | Sour | 2 | 0.1% Citric Acid | Soft Drinks, Catsup |
| 3 | Bitter | 1 | 0.05% Caffeine | Whole Peanuts, |
| | | | | Milk Chocolate |
| 4 | Bitter | 2 | 0.10% Caffeine | Beer |
| 5 | Sweet | 1 | 5% Sucrose | Peanut Butter, |
| | | | | Unsweetened Juice |
| 6 | Sweet | 2 | 10% Sucrose | Soft Drinks, Vanilla |
| | | | | Ice Cream |

*Aqueous solutions [wt/vol % or wt/wt %]

Two commercially available nutritional products, AMIN-HEPA™ (distributed by Societe Dietetique Francaise de Formulation et de Fabrication, Doullens, France) and Aminoleban® EN (distributed by Luen Cheong Hong LTC, Hong Kong) were purchased and reconstituted per the printed instructions. The liquid nutritional composition of Example I was manufactured with six different flavors as described in Example I.

The AMIN-HEPA™ sample was prepared by reconstituting one 55 gm pouch of powder in 150 ml of room temperature water in a blender on low speed. The Aminoleban® EN sample was prepared by reconstituting 50 gm of powder in 180 ml of room temperature water in a blender on low speed. The pineapple flavor mix pouch was then added to the Aminoleban® EN blended product. Containers of the liquid nutritionals of the instant invention (experimental) were shaken prior to evaluation at room temperature.

The compositions were evaluated and the results of the organoleptic test scoring are also set forth in Table 11.

TABLE 11
Taste Test Scores for Sour, Bitter, Brothy and Sweet

| TASTE TEST SCORE | Sour | Bitter | Brothy | Sweet |
|---|---|---|---|---|
| Aminoleban ® EN pineapple | 1½ | 1½ | 1 | 2 |
| Amin-Hepa ™ | 1 | 1 | 1 | 1½ |
| Experimental spicy vanilla | 1 | 1 | 0 | 1½ |
| Experimental vanilla cookie | ½ | ½ | 0 | 1½ |
| Experimental toasted rice | ½ | 0 | 0 | 1½ |
| Experimental orange | 1 | 1 | 0 | 1½ |
| Experimental melon | 1 | 1 | 0 | 1½ |
| Experimental cherry almond | 1 | 1 | 0 | 1½ |

The highest bitter, sour and sweet values were assigned to Aminoleban® EN. Interesting that even with a sweetness of a 2, Aminoleban®EN still had bitterness of 1½. Typically, sweet notes are added to help mask the bitter notes. Amin-Hepa™ and a few flavors of the experimental formulation had similar sour, bitter and sweet notes. However, Amin-Hepa™, as well as Aminoleban®EN, had an additional brothy note that was not detected in any of the experimental formulas. The vanilla cookie and toasted rice flavored experimental formulas had low bitter notes while being less sweet.

We claim:

1. A nutritional product adapted for use in patients with liver disease, comprising an amino-nitrogen component, a fat component and a carbohydrate component, wherein said amino-nitrogen component provides about 12 to about 20% of the total caloric content of the product and comprises less than or equal to 30 wt/wt % BCAA.

2. The nutritional product according to claim 1 wherein said amino-nitrogen component provides a BCAA to AAA ratio of from about 1.5:1 to about 5:1.

3. The nutritional product according to claim 1 wherein said amino-nitrogen component provides 12 to about 18% of the total caloric content.

4. The nutritional product according to claim 3 wherein said amino-nitrogen component provides about 14 to about 18% of the total caloric content.

5. The nutritional product according to claim 1 wherein the amino-nitrogen component comprises from about 20 to about 27 wt/wt % of BCAA.

6. The nutritional product according to claim 1 wherein the amino-nitrogen component comprises no more than about 15 wt/wt % of AAA.

7. The nutritional product according to claim 2 wherein the ratio of BCAA to AAA is from about 1.5:1 to about 4:1.

8. The nutritional product according to claim 1 wherein said fat component provides about 25 to about 35% of the total caloric content of the product and comprises a weight ratio of ω-6 fatty acids to ω-3 fatty acids from about 1.5:1 to about 5:1.

9. The nutritional product according to claim 8 wherein said weight ratio of ω-6 fatty acids to ω-3 fatty acids from about 1.5:1 to about 3:1.

10. The nutritional product according to claim 1 wherein said fat component comprises from about 15 to about 30% high oleic sunflower oil, about 15 to about 30% MCT, about 20 to about 35% borage oil, about 15 to about 30% sardine oil, and about 3 to about 6% fungal oil, based on the weight of the fat component.

11. The nutritional product according to claim 10 wherein said fat component further comprises from about 3 to about 5% soy lecithin based on the weight of the fat blend.

12. The nutritional product according to claim 1 in which said nutritional further comprises from about 0.1 to about 0.4 gm/100 Kcal of GLA.

13. The nutritional product according to claim 1 in which said nutritional further comprises from about 0.1 to about 0.4 gm/100 Kcal of EPA.

14. The nutritional product according to claim 1 in which said nutritional further comprises from about 0.03 to about 0.3 gm/100 Kcal of AA.

15. The nutritional product according to claim 1 in which said nutritional further comprises from about 0.03 to about 0.3 gm/100 Kcal of DHA.

16. The nutritional product according to claim 1 in which said nutritional further comprises from about 0 to about 1.5 gm/L of diphosphatidyl choline.

17. The nutritional product according to claim 1 wherein said carbohydrate component provides about 45 to about 65% of the total caloric content of the product.

18. The nutritional product according to claim 17 in which said nutritional further comprises from about 5 to about 15 gm/L of FOS.

19. The nutritional product according to claim 1 in which said nutritional further comprises an antioxidant system, said antioxidant system comprises beta-carotene from about 390 to about 1200 ug/L, vitamin E from about 195 to about 600 IU/L, vitamin C from about 195 to about 600 mg/L and selenium from about 20 to about 90 ug/L.

20. A method for providing nutrition to an individual with liver disease, comprising administering the nutritional product according to claim 1.

21. A method for correcting the nutritional deficiencies of a hepatic patient by adiministering the nutritonal product according to claim 1.

22. A method for attenuating the progression of liver disease by administering the nutritional product according to claim 1.

23. A method for improving liver function by administering the nutritional product according to claim 1.
